(19) United States
(12) Patent Application Publication
(10) Pub. No.: US 2018/0100848 A1
(43) Pub. Date: Apr. 12, 2018

(54) DEVICE AND METHOD FOR MONITORING MATERIAL PROPERTIES OF PLASTICS

(71) Applicant: Dr. Collin GmbH, Ebersberg (DE)

(72) Inventor: Friedrich KASTNER, Grieskirchen (AT)

(21) Appl. No.: 15/728,874

(22) Filed: Oct. 10, 2017

(30) Foreign Application Priority Data
Oct. 10, 2016 (EP) 16002181.2

(51) Int. Cl. G01N 33/44; G01N 21/84; G01N 11/00; G01N 21/95; G01N 21/359; G01N 21/27; G06T 7/00; G06T 7/90; B29C 47/92; B29C 47/00; B29C 47/14; B29C 55/18

(52) U.S. Cl. CPC G01N 33/442; B29L 2007/008; G01N 11/00; G01N 21/95; G01N 21/359; G01N 21/27; G06T 7/0004; G06T 7/90; B29C 47/92; B29C 47/0021; B29C 47/0057; B29C 47/14; B29C 55/18; G06T 2207/30164; B29C 2947/922; B29C 2947/92228; B29C 2947/92257; B29C 2947/92409; G01N 21/8422

(57) ABSTRACT

The invention relates to a device and a method for inline quality checking for plastics during the production process, comprising at least two different measuring devices, wherein the device has a unit for sampling plastic melt from the production extruder, a unit for moulding a flat film from this plastic melt, and units for transporting the flat film to the measuring devices and for finishing the flat film.

The invention relates to a device for monitoring material properties of plastics during the production method.

Methods and corresponding devices for checking and measuring the physical parameters and material properties of plastics are known. Thus, for example, in the case of plastic production, in particular in the case of film production, a pressure filter test is used to ascertain the dispersion of additives or contaminants of a polymer, a rheometer is used to determine the viscosity properties, an optical film inspection, colour measurements, near infrared spectroscopy (NIR), Fourier transform infrared spectrometry, mass spectrometry, or mechanical strength measurements, for example, tension and elongation measurements, scratch resistance of the surface and the like are used to determine the properties of the product. These study methods are largely standardized.

In general, these studies are carried out after taking samples during the production process and/or after the production of the product off-line in various measuring devices, usually externally in analysis and quality control laboratories. The corresponding results are then usually available only after completion of the production process, whereby a reaction to quality deviations during the production process is nearly impossible. The quantity of flawed products, which do not correspond to the quality requirements, is thus increased. These discards then have to be sorted out and/or reprocessed.

It is therefore advantageous to analyse the relevant properties of the product to be produced and/or the raw material or the precursor material during the production process, to be able to engage in the production process in a timely manner and therefore reduce the quantity of finished product which does not meet the quality requirements.

The object of the invention was to provide a device and a method for quality monitoring and quality judgment for plastics, which enables certain parameters and properties of the product and the precursor product to be determined in-line, i.e., already during the production process, to be able to react quickly to deviations from the target state and therefore substantially avoid the production of flawed products.

The subject matter of the invention is therefore a device for in-line quality checking for plastics during the production process, comprising at least two different measuring devices selected from the group of measuring devices for determining the rheological properties, measuring devices for colour measurement, measuring devices for detecting inclusions and gel particles and the like, measuring devices for judging the optical properties, and measuring devices for determining the mechanical parameters, characterized in that the device has a unit for sampling plastic melt from the production extruder, a unit for moulding a flat film from this plastic melt, and units for transporting the flat film to the measuring devices and for finishing the flat film.

A further subject matter of the invention is a method for quality checking for plastics during the production process, characterized in that a defined quantity of plastic melt is conducted directly from a production extruder into the device for quality checking and subsequently moulded into a flat film, whereupon the flat film is studied and judged with the aid of the measuring devices situated in the device.

By sampling the melt in-line directly from the production extruder and checking the flat film moulded from the melt still during the production process, quality deviations can be reacted to immediately and quickly and unnecessary discards of product can thus be avoided.

The device according to the invention has a unit for taking a sample of the plastic melt from the production extruder. In the simplest case, this unit is implemented by an adapter, which has a borehole in the simplest case, to which a melt line to the device according to the invention is attached, or by a borehole directly into the cylinder housing of the discharge region of the extruder.

However, with these solutions, one accepts the disadvantage that due to edge effects, the flow velocity at the edge is very low, and therefore one runs the risk of correlating samples which are actually already changed, degraded, or chronologically no longer relevant with the material flow.

To avoid this, according to the invention adapters or intermediate parts can be installed, which ensure that melt samples are taken over the entire or at least from or in the middle of the main flow. In one simple embodiment, the sampling opening is simply a pipe end here, which is directed into the melt flow.

However, a displacement body can also likewise be introduced into the melt flow, which is embodied such that a sampling point for the melt flow is located in this displacement body. The sampling point can either be a borehole in this case, which conducts the melt flow inside the displacement body from the extruder, or a melt channel, which discharges the melt flow along the surface of the displacement body. In both cases, it is necessary for the sampling point to be formed so that an independent melt discharge results because of the pressure and flow conditions prevailing in the extruder. The embodiment as a melt channel is advantageous insofar as a representative melt sample flowing radially via the cylinder of the extruder can be taken.

A simple embodiment of such a displacement body can be represented by a fluidically optimized spoke which extends diagonally through the cylinder of the extruder and has a melt channel attached on the end face. Three or more spokes can also likewise be embodied in a star shape, however. In both embodiments, it is advantageous if the displacement body is positioned like a torpedo against the flow direction in the extruder.

The device according to the invention is coupled to a side strand of the extruder using the adapter and a defined quantity of plastic melt is possibly sampled from the extruder via a melt pump after opening of a valve. In this case, the pressure loss is already measured by means of a rheometer, consisting of a measuring nozzle having defined cross section, over a specific measuring distance. The viscosity of the melt can be determined from the pressure loss.

Subsequently, a flat film is produced from the melt by means of a sheet die and a casting roller to ascertain further properties. It is particularly important in this case that the film has homogeneous properties (thickness, colour, etc.) to ensure an optimum check later. Optimum cooling, but also the condition of the surface of the casting roller, are therefore essential.

In the simplest case, the surface is embodied as glossy. To ensure the detachment of various materials, the roller can be produced having coatings such as glossy chrome, anti-adhesive layers such as Teflon, or other common anti-adhesive layers or combinations thereof.

To provide better conditions for special studies, however, the roller surface can also be formed having other textures or textures which are different in the longitudinal and transverse directions, such as matte points, embossments, different degrees of gloss-matte, patterns, and the like. The goal of these structures is, for example, to prepare the surface for subsequent studies, such as olfactory studies (odours) or visual studies.

The film thus produced in the device is supplied to a visual check.

For this purpose, inclusions and gel particles are detected by means of a high-resolution area camera or line camera and downstream high-performance computer and analysed on the basis of suitable analysis software, which recognizes the size, shape, and location of the inclusions and flaws and can assign them into classes. The recognition of the flaws takes place in this case via grey scale or colour changes of individual pixels of the recorded image.

The size of the inclusions and flaws can be defined in the simplest case via the number of the incorporated pixels, but can also be expressed via the area of the enclosing circle or enclosing rectangle of the two extreme values (length, width). In the latter case, a statement may also be made about the shape of the flaw, since this relates to the aspect ratio of the flaw in this case.

The illumination of the film is expediently performed via a dome light and two ring lights arranged in parallel above and below the film to be able to measure both in the reflection mode and also in the transmission mode. However, line, bar, or area illumination units can also be used. The illumination units are attached in an encapsulation which is easy to remove, to prevent scattered light from the outside.

White light is typically used for the illumination, however, depending on the camera and illumination type, different colours or combinations of colours in incident and transmitted light can also be used. In addition, illumination units which are arranged at different angles in relation to the film surface can help to facilitate the flaw recognition.

Furthermore, filters such as linearly or circularly polarizing filters can be used in front of the camera and/or behind the camera, for example, to to be able to better detect flaws or to supplement the analysis of the materials by the optical properties thereof.

In one special case, UV illumination units (in various wavelengths) can also be used, for example, to study whether the material itself or contaminants fluoresce in specific spectral ranges. To measure all of these properties, one or multiple illumination and/or camera units can be combined in parallel or in series.

Furthermore, the plastic film is subjected to a colour measurement. In this case, the film is illuminated using a daylight lamp or in succession using different colours and the light originating from the film is split into various wavelength ranges. The colorimetric data are computed by means of a sensor or a sensor system.

The display of the ascertained colour value is preferably performed in the L*a*b colour space, which is known to a person skilled in the art. However, other colour spaces such as the RGB, CMYK, or Pantone colour space can also be used.

In a further step of the examination, the film is subjected to an NIR measurement. This method enables foreign materials in the plastic film to be recognized. This is important in particular in the case of recycling plastics, to ensure a uniform product quality.

In this case, a multispectral sensor is expediently used, which is very cost-effective in contrast to conventional NIR spectrometers.

The sensor operates in selected light wave ranges and measures the absorption of the light in this narrow wavelength range. Contaminants or changes in the composition, for example, can be concluded in this case via the absorption rate. Since the sensor only operates in a narrow light spectrum, it is calibrated beforehand especially to the material to be studied.

Before the mechanical study, the film is cut to a defined width. The remaining film strip is measured directly online, for example, solely mechanically via a scanning head or optically (for example, IR) or capacitively or by radiation (x-ray radiation) for its thickness and subsequently stretched or elongated via two roller pairs. The torque required for the stretching is measured and an equivalent to the modulus of elasticity (E-modulus) is computed therefrom.

The stretching can be executed in this case via two roller pairs, which run at different speeds, or via a combination of two roller pairs which run identically, and a stretching roller arranged in between, which changes the web path during a measurement.

If the second roller pair is set into an accelerated movement, a monoaxial tensile experiment having continuously increasing elongation, for example, as is carried out in a laboratory experiment, can thus also be readjusted. If necessary, all common optical measuring methods can additionally be used in the device according to the invention, for example, gloss measurement, haze measurement, UV-VIS, ellipsometry, x-ray fluorescence, and the like.

However, combined analyses, for example, scratch measurement with visual recognition of the defect, or further mechanical characteristic values such as tear propagation test, constriction behaviour, or temperature-dependent tensile properties can also be provided in the device according to the invention.

A test for odours is also particularly important for the recycling field. Since heretofore actual odour sensors unfortunately do not exist, a simple measurement can be implemented via a simple gas chromatograph in a module, however.

For this purpose, the film is strongly heated, at best already during the production of the film, i.e., during the casting process, or once again later, for example, by infrared, hot air, or via a contact roller, and the resulting vapours are suctioned off and conducted into a gas chromatograph. If a very short measuring column is used, for example, 10 cm, a rapid measurement can be implemented, to thus enable detection of various ingredients, which can be odorous or also neutral. In this case, the longer the column, the better the resolution and the longer the measuring time.

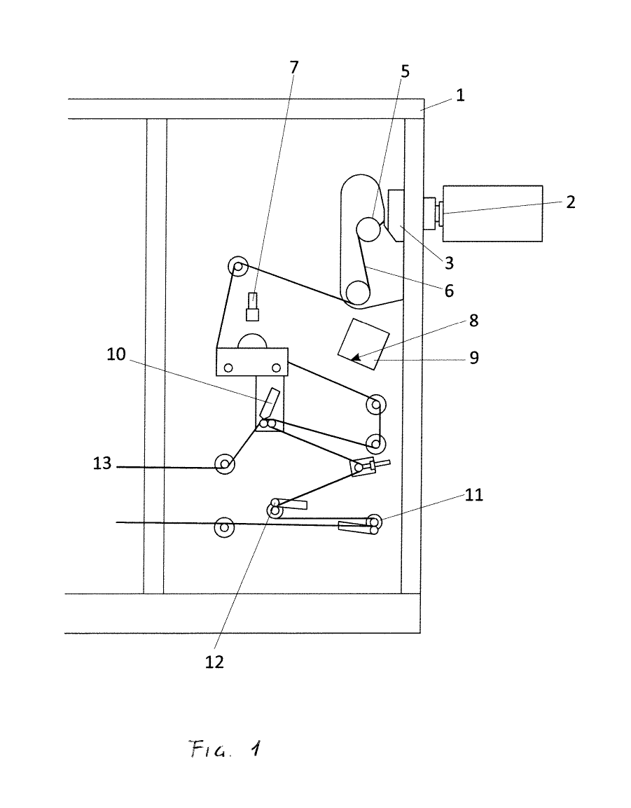

FIG. 1 shows a device according to the invention.

The device consists of a housing 1, which has a unit 2 for sampling the plastic melt, which is transported through a measuring nozzle for determining the rheological properties, in particular the viscosity of the melt, into the interior of the housing of the device.

Subsequently, the plastic melt is moulded by the sheet die 3 and a casting roller 5 into a flat film 6 and guided via deflection rollers to the first visual check by means of an area camera 7 for recognizing inclusions and gel particles. The film is conducted in this case through a housing to exclude scattered light and is illuminated using a dome light and ring lights.

Subsequently, the flat film is guided further to the colour measurement 8 and NIR measurement 9 by means of a multispectral sensor.

Before the subsequent study, the film is cut into strips by an edge cut 10. The middle strip preferably has a width between 20 and 25 mm, to thus correspond to the standard test specimen from the film tensile experiment (ISO 527-3), but can also be embodied wider or narrower. The middle strip is stretched between two roller pairs 11, 12 to ascertain the modulus of elasticity. The two edge strips 13 are not used for the tensile experiment.

All measuring devices and the processing units are situated in a space-saving manner in the housing. The required parameters of the plastic or the plastic film can thus be ascertained directly, still during the production at one location, and the production process can be engaged in if necessary. The quantity of material not corresponding to the quality criteria is thus significantly reduced.

In addition, measurement points which are situated at different locations along the production device are not necessary due to the single sampling at a single point of the production process.

1. Device for in-line quality checking for plastics during the production process, comprising at least two different measuring devices selected from the group of measuring devices for determining the rheological properties, measuring devices for colour measurement, measuring devices for detecting inclusions and gel particles and the like, measuring devices for judging the optical properties, and measuring devices for determining the mechanical parameters, studying the olfactory parameters, wherein the device has a unit for sampling plastic melt from the production extruder, a unit for moulding a flat film from this plastic melt, and units for transporting the flat film to the measuring devices and for finishing the flat film.

2. Device according to claim 1, wherein the device is coupled via an adapter to a side strand of the extruder and the sampling of the plastic melt occurs after opening of a valve.

3. Device according to claim 1, wherein the sampling of the plastic melt is performed through a borehole into the cylinder housing of the output region of the extruder.

4. Device according to claim 1, wherein the sampling of the plastic melt is performed by a displacement body, which is positioned in the melt flow and around which the melt is conducted to a borehole in the cylinder housing of the extruder.

5. Device according to claim 1, wherein the device has a sheet die and a casting roller for moulding a flat film.

6. Device according to claim 1, wherein the device comprises two roller pairs for stretching the flat film.

7. Device according to claim 1, wherein the device comprises a measuring nozzle for determining the rheological properties.

8. Device according to claim 1, wherein the device comprises an area camera for recognizing inclusions and gel particles in the flat film.

9. Device according to claim 1, wherein the device comprises a multispectral sensor for NIR measurement.

10. Device according to claim 1, wherein the device comprises an illumination unit and a sensor for colour measurement.

11. Method for quality checking for plastics during the production process, wherein a defined quantity of plastic melt is conducted directly from a production extruder into the device for quality checking and subsequently moulded into a flat film, whereupon the flat film is studied and judged with the aid of the measuring devices situated in the device.
